(19) United States
(12) Patent Application Publication
(10) Pub. No.: US 2019/0336098 A1
(43) Pub. Date: Nov. 7, 2019

(54) A SYSTEM AND METHOD FOR DETERMINING FAT AND LEAN BODY TISSUE COMPONENTS

(71) Applicant: KONINKLIJKE PHILIPS N.V., EINDHOVEN (NL)

(72) Inventors: Jingping Xu, Shanghai (CN); Cong Tian, Shanghai (CN); Lin Li, Shanghai (CN)

(21) Appl. No.: 16/322,696

(22) PCT Filed: Aug. 1, 2017
(86) PCT No.: PCT/EP2017/069389

§ 371 Date: Feb. 1, 2019

(30) Foreign Application Priority Data
Aug. 8, 2016 (CN) PCT/CN2016/094012
Sep. 20, 2016 (EP) 16189603.0

(51) Int. Cl. A61B 8/08; A61B 8/00

(52) U.S. Cl. CPC A61B 8/0858; A61B 8/4488; A61B 8/485; A61B 8/54

(57) ABSTRACT

A system is for determining fat and lean body tissue components of body tissue. A propagating wave is provided into the body tissue. A first set of normally directed reflections of the propagating wave is received at a first measurement site and a second set of angled reflections of the propagating wave is received from the body tissue at a second measurement site. A fat body tissue depth and optionally also a lean body tissue depth are obtained based on the times at which reflections of the first and second sets are received. This arrangement requires only two receivers in order to resolve the depth of the fat and lean body tissue constituents. The propagation paths are simplified by arranging the transceiver to direct a wave normally into the tissue.

### FIELD OF THE INVENTION

This invention relates to determination of the fat tissue component and lean tissue component of a subject, for example to determine a fat to lean ratio or total mass of body fat tissue. This fat to lean ratio may be a thickness ratio of fat and muscle thickness under the skin, or a ratio of fat mass and muscle mass. One area of interest is for monitoring the body composition of an expectant mother during pregnancy. Other areas of interest are for applications relating to the elderly population, or as part of fitness or diet monitoring.

### BACKGROUND OF THE INVENTION

Expectant mothers often experience difficulties in deciding the correct amount and type of food to eat, in order to ensure the wellbeing of their child while preventing excessive weight gain of the mother herself. In particular, it is of interest to know if weight gain is partly attributable to an undesired level of increase in fat tissue, or only attributable to the expected weight gain during pregnancy. An expectant mother may desire to manage her own weight during pregnancy so that she can get back to her shape after the delivery.

Magnetic resonance imaging (MRI) and computer tomography (CT) scanning are the standard technologies for the quantitative assessment of the fat constituent of the overall body mass. However, none of these technologies is suitable for a consumer device where repeatable measurements (for example fat thickness) are desired on a daily basis.

The maternal body composition exhibits dynamic changes during pregnancy to support the development of the fetus from conception through to birth. These changes can be reflected as gestational weight gain (GWG), which includes the weight gains in maternal fat mass (FM) and fat-free mass (FFM) and also the fetus, placenta and amniotic fluid. If these body composition changes could be measured, this would provide a useful tool for monitoring the body changes during pregnancy.

One known way to analyze body tissue composition is to use bio-impedance analysis (BIA). The principle behind BIA is to measure human tissue response to electrical current based on different electrical properties of human tissue components. However, it is not applicable for measurement of body compositions in pregnant women because there is a working assumption that the ratio of extracellular vs. intracellular water remains constant. This is violated due to the physiological change during pregnancy. BIA is also unable to distinguish between the maternal fat and the fetal and supporting tissues.

In clinics, body fat mass and lean mass can be estimated by empirical equations relating to subcutaneous fat thickness, in certain regions (e.g. biceps, triceps, etc.), and gestation weight. Such a method is described in Widen EM, Gallagher D. “Body composition changes in pregnancy: measurement, predictors and outcomes”, European Journal of Clinical Nutrition, 2014, 68: 643-652. However, such methods are not applicable at home, since professional healthcare instruments are needed, such as ultrasound equipment or physician's calipers, as well as extensive training to technicians or other operators.

For example, a full-functional ultrasound imaging system requires specific training to be able to identify fat tissue from muscle and other softer tissue. For non-experienced users, identifying fat tissue from an ultrasound image is challenging, since ultrasound images of the region of interest are highly noisy. The fat and muscle boundaries can be at various depths among different people. The identification of the fat boundary line is therefore complicated due to the shapes and image texture of fat and muscle boundaries both being highly variable.

An ultrasound imaging system is generally also too expensive for personal use at home. There are also difficulties in guiding users (particularly inexperienced users) to position the ultrasound probe correctly on the skin. An applied pressure may also influence the thickness of the fat in the measurement.

There are other techniques for analyzing body composition changes, such as underwater weighing. This is unable to distinguish maternal weight composition from a fetal weight part and supporting tissues, or to provide accurate discrimination between fat tissue and lean tissue.

There is therefore a need for a system and method which enables body composition monitoring in a simple manner and suitable for use at home.

### SUMMARY OF THE INVENTION

The invention is defined by the claims.

According to examples in accordance with an aspect of the invention, there is provided a system for determining fat and lean body tissue components of body tissue, comprising:

a transceiver for transmitting a or more propagating wave normally into the body tissue, and receiving a first set of normally directed reflections of the propagating wave from the body tissue at a first measurement site;

a receiver for receiving a second set of the propagating wave from the body tissue at a second measurement site, which is displaced away from the first measurement site;

a timing arrangement for determining or estimating the times at which reflections of the first and second sets are received; and

a processor, which is adapted to:
control the transceiver;
receive a distance between the first measurement site and the second measurement site;
   and
process the times determined or estimated by the timing arrangement as well as and the distance between the transceiver and the receiver the distance received, thereby to derive a fat body tissue depth.

This arrangement formulates two different propagation paths to resolve the depth of the fat tissue and lean tissue components. The two propagation paths are simplified by arranging the receiver and/or the transceiver to receive the reflections at two different measurement sites. This generates a first reference set of reflections received at a first set of time points at the first receiver at the first measurement site and a second reference set of reflections received at a second set of time points at a second measurement site, where each reference set of reflection comprise the echoing waves propagating through one path and reflected at two reflective interfaces between different body tissues, as a first interface between the fat and muscle and a second interface between the muscle and the bone. With the distances between the first measurement site and the second measurement site provided and two sets of the time points of received reflections of the propagating waves, it enables determinations of different speeds of wave travel in the lean and fat tissue as well as the different depths. Thus, four different parameters may be extracted even though only two measurement sites are used. The speeds of waves propagating in tissues are assumed to be user specific according to the different compositions or properties of the fat/lean tissue of different users.

In one embodiment, the transceiver is adapted to transmit a first propagating wave at the first measurement site and the receiver is displaced from the transceiver along the body tissue for receiving the second set of angled reflections of the first propagating wave.

The second measurement site may be formulated by placing an individual receiver along the body tissue for receiving the second set of angled reflections of the first propagating wave due to the scattering. One propagating wave is transmitted at the first measurement site and two set of reflections of the one propagating wave will be received by the transceiver and receiver respectively, which are one set of normal reflections and one set of angled reflections with a longer propagation path. The two paths, and hence two sets of timings, enable the different speeds of wave travelling in the lean and fat tissue to be resolved as well as the different depths. Thus, four different parameters may be extracted even though only two measurement sites are used.

The transceiver and receiver may be at a fixed distance or the distance may be varied between different measurements.

The system may also obtain a lean body tissue depth.

In one embodiment, the transceiver is adapted to transmit a first propagating wave at the first measurement site and transmit a second propagating wave at the second measurement site, and the receiver is the transceiver displaced at the second measurement site for receiving the second set of normal reflections of the second propagating wave.

The two propagation paths are formulated by pressing the transceiver in the direction that is perpendicular to the tissue layer, which creates two positions of the transceiver before and after pressing, namely, the first measurement site and the second measurement site. Two propagating waves will be transmitted respectively at the first measurement site and the second propagating wave by the transceiver at the second measurement site. Further, the transceiver is arranged to receive a set of normal reflections of the first propagating wave at the first measurement site and to receive a set of normal reflections of the second propagating wave at the second measurement site. A time interval shall be allocated to avoid the overlapping of the first and second set of reflections received. The durations of the all reflections will be recorded by the time arrangement. In this way, the hardware is simpler and no additional receiver is needed. It will be more cost effective than the solution proposed earlier.

The distance between the first measurement site and the second measurement site may be determined by a deformation sensor attached to a contact area of the transceiver with the body tissues during the measurement.

A deformation sensor may be attached to the contact area of the probe with the tissue layer. During the movement of the transceiver caused by the external force applied, the deformation sensor is arranged to make the measurements of the distance between the two positions, upon which the transceiver transmits and receives two set of reflected echoes respectively. In this way, the user is free to operate the transceiver without any restriction of the movement during the measurement.

The propagating wave may comprise an ultrasound wave.

The transceiver is then preferably a high frequency and preferably wideband ultrasound transducer. Other waves may be used, such as infrared or electromagnetic waves.

In a simplest form, the transceiver may have only one transmitting and receiving element. However, it may comprise more elements to constitute an ultrasound transducer array.

The transceiver may be considered to be at a single measurement site so it is intended to capture a set of timing values for one propagation path. However, it may comprise an array device. This does not complicate the signal processing, in that the array device is used as a single unit for sending ultrasound waves into the tissue to be measured (as the source) and for receiving echo data (as the sensor).

The transceiver may comprise a transducer which emits ultrasound waves at more than 1 MHz.

In a simplest form, the receiver may have only one receiving element. However, it may also comprise an ultrasound receiver array.

Again, the receiver may be considered to be at a single measurement site so it is intended to capture a set of timing values for one propagation path. However, it may comprise an array device. This does not complicate the signal processing, in that the array device is used as a single unit sensor.

The receiver may comprise a transceiver which is operated only in a receiving mode.

This means the two devices may be identical, reducing the cost and complexity of the system. They may both be ultrasound transducer arrays, or they may be portions of a shared large ultrasound transducer array which extends between the two locations at which measurements are taken, but is only operated at the two measurement locations.

The system may further comprise a reference membrane or patch over the transceiver such that it is in the wave path between the transceiver and the body tissue.

This reference membrane for example has known reflection and/or attenuation characteristics. The knowledge of these characteristics for example enables compensation for temperature and humidity.

The processor may be adapted to determine whether or not the body tissue has a high water content indicative of water retention. This can be achieved based on a comparison of the observed attenuation characteristics in the body tissue and the known attenuation characteristics of the reference membrane. If the attenuation deviates significantly from that which is known to take place in the reference membrane, it can be judged that the body tissue has a very different attenuation characteristic to the reference membrane, caused by water retention.

The distance between the transceiver and the receiver may be in the range 3 cm to 5 cm. This enables a compact portable system to be formed.

The processor may be adapted to derive an overall body fat measurement. This may be achieved based on a regression analysis having as input the fat tissue and lean tissue depths, for example at different body locations. It may also have as input the total weight of the subject, their height, gender etc.

This system may be for monitoring a fat to lean body tissue ratio of an expectant mother during pregnancy.

Examples in accordance with another aspect of the invention provide a method for determining fat and lean body tissue components of body tissue, comprising:

transmitting a or more propagating wave normally into the body tissue, and receiving a first set of normally directed reflections of the propagating wave from the body tissue through a first propagation path at a first measurement site;

receiving a second set of reflections of the propagating wave from the body tissue through a second propagation path at a second measurement site which is displaced from first measurement site;

determining or estimating the times at which reflections of the first and second sets are received; and

receiving a distance between the first measurement site and the second measurement site;

processing the times determined or estimated by the timing arrangement (24) and the distance received, thereby to derive a fat body tissue depth.

The method may also derive a lean body tissue depth.

The propagating wave may comprise an ultrasound wave. Transmitting the propagating wave into the body tissue may be carried out through a reference membrane.

The processing employed in the system of the invention may be implemented at least in part in software.

### BRIEF DESCRIPTION OF THE DRAWINGS

Examples of the invention will now be described in detail with reference to the accompanying drawings, in which:

FIG. 1 shows how the weight distribution of an expectant mother varies during pregnancy;

FIG. 2 show a system for discriminating between a fat and lean body tissue;

FIG. 3 identifies the acoustic paths in the system of FIG. 2 in order to show how the timing calculations are performed;

FIG. 4 shows a method for discriminating between fat and lean body tissue; and

FIG. 5 shows a general computer architecture suitable for implementing the processing performed within the system of FIG. 2;

FIG. 6 illustrates a simplified system with identified acoustic paths in order to show how the timing calculations are performed.

### DETAILED DESCRIPTION OF THE EMBODIMENTS

The invention provides a system for determining fat and lean body tissue components of body tissue. A propagating ultrasound wave is provided into the body tissue. A first set of normally directed reflections of the propagating wave is received at a first measurement site and a second set of angled reflections of the propagating wave is received from the body tissue at a second measurement site. A fat body tissue depth and optionally also a lean body tissue depth are obtained based on the times at which reflections of the first and second sets are received. This arrangement requires only two receivers in order to resolve the depth of the fat and lean body tissue constituents. The propagation paths are simplified by arranging the transceiver to direct a wave normally into the tissue.

The invention is of particular interest for expectant mothers who wish to monitor their weight gain, although the invention may be applied generally to body tissue examination.

Note that the system does not necessarily output a ratio value or separate parameters for the fat tissue and the lean tissue. It may have as output only information about the fat body tissue, having first discriminated between lean body tissue and fat body tissue.

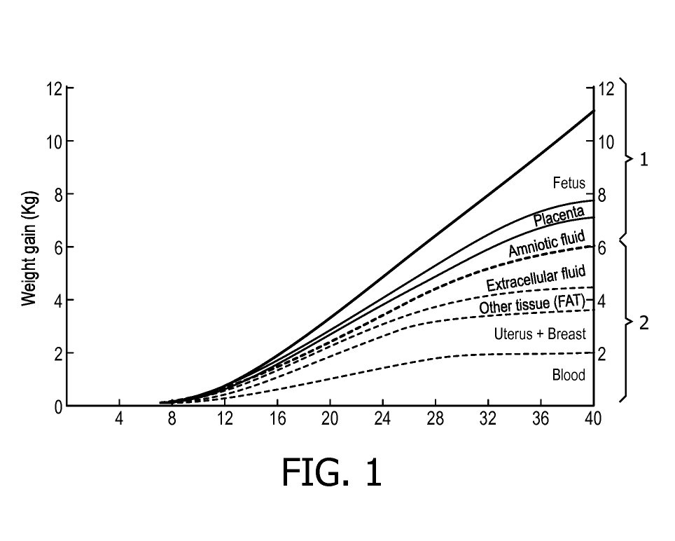

FIG. 1 shows how the weight distribution of an expectant mother varies during pregnancy. The y-axis shows the weight gain in kg and the x-axis is the time in weeks from conception. There is typically a generally linear overall weight gain from 8 weeks through to the delivery. FIG. 1 shows the contribution of different components to this weight gain, between the mother 1 and the fetus 2. The fetal load is the combination of the fetus, placenta and amniotic fluid. This is taken from Pitkin, “Nutritional support in obstetrics and gynecology”, Clinical Obstetrics and Gynecology[J] 1976, 19(3): 489-513.

The graph shows a pregnant woman with 11 kg gestational weight gain. The total fetal load is around 5 kg. The weight increase of the uterus and breast is around 3.5 kg and the total fat change of the mother is around 1 kg, which includes the fat gain from the thighs, hip, abdomen, and arms.

One way for an expectant mother to be able to distinguish between maternal weight gain and weight gain resulting from the fetus would be to measure the body tissue fat component and the body tissue lean component. This would then provide an indication of changes in the maternal body tissue constituency.

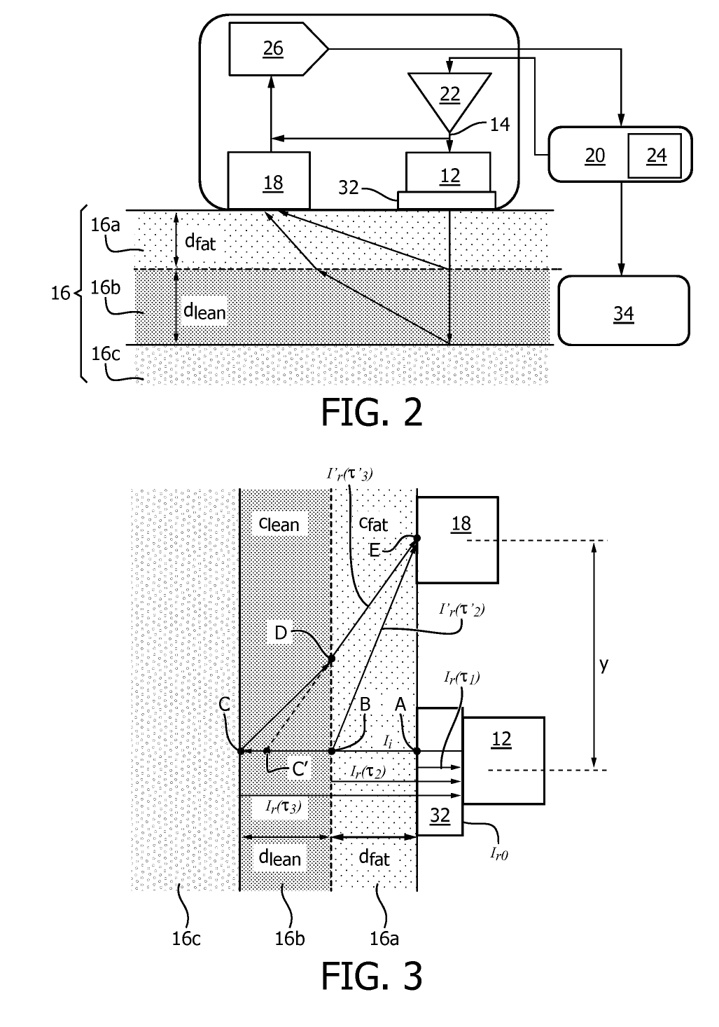

FIG. 2 shows a system 10 for determining (i.e. distinguishing between) a fat body tissue component and a lean body tissue component, comprising a transceiver 12 having a transmitter for transmitting a propagating wave 14 into the body tissue 16 and a first receiver for receiving a first set of normally directed reflections of the propagating wave from the body tissue at a first measurement site.

The body tissue comprises a fat body tissue layer 16a, and a lean (muscle) body tissue layer 16b, lying over the bone 16c. Due to the limited distance between the transmitter and receiver, the depth of the fat body tissue layer 16a is considered to be constant, and the depth of the lean (muscle) body tissue layer 16b is considered to be constant.

A second receiver 18 is displaced from the transceiver 12 along the body tissue for receiving a second set of angled reflections of the propagating wave from the body tissue at a second measurement site.

A controller 20 controls the transceiver 12 via a transmission pulse circuit 22. It also includes a timing arrangement 24 for detecting the times at which pulses are received at the first and second receivers. The receiver signals are provided to an analog to digital converter 26 which provides digital signals to the processor 20. The analog to digital converter may be combined with other filtering and signal processing functions. The controller 20 includes a processor 24 for processing the times determined by the timing arrangement as well as the distance between the two receivers, thereby to derive a fat body tissue depth dimension d_{fat} and a lean body tissue depth dimension dₗₑₐₙ.

The system 10 may be implemented as a hand held probe. The transceiver 12 and the receiver 18 may each comprise a high-frequency (>1 MHz) wideband ultrasound transducer, although only the receiver function of the second transducer is needed. By way of example, a central frequency may be around 5 MHz to achieve a good spatial resolution and enough imaging depth. The imaging depth may be in the range 8 cm to 15 cm.

The (or each) transducer for example comprises a piezoelectric disk transducer comprising an array of piezoelectric elements having a matching layer at the ultrasound output face of the transducer and a damping layer at the opposite face. Electric voltage pulses are applied to the piezoelectric elements to cause an ultrasound pulse to be emitted from the output face. A reflected echo is converted by the same piezoelectric process into return echo pulses. The thickness of the disk element corresponds to half a wavelength. For example, a thickness of 0.2 mm to 1 mm corresponds to a frequency of 10 MHz to 2 MHz respectively (with a propagation speed in the piezoelectric material of 4 to 6 mm/μs).

There may be an ultrasound array transducer with a small number of transducer elements. A first one or a first set of these transducer elements may be used for transmitting and receiving at the first measurement site and a physically displaced second one or second set of transducer elements may be used as the second receiver. The system is applied to a selected body tissue region, such as the biceps or triceps.

The transceiver 12 also has a reference membrane in the form of a patch 32 between the transceiver output and the body tissue 16. The transceiver 12 also has a transmit/receive switch. This is optional for the receiver 18 as it is only needed to be operated in a reception mode.

The controller 20 may be remote from the hand held part of the device, for example implemented as a computer or portable device, such as smart phone, to process the incoming signals and provide an output representing the fat and lean body tissue content.

The output may be a depth ratio, or this may be converted to a mass ratio or else a total fat tissue mass. The conversion from the depth information which is obtained directly by the measurement into other parameters (such as overall fat content or a ratio) may be obtained using a regression equation.

An output device such as a display 34 provides the output data. The output device can be in any suitable form, including projection onto the abdomen of the expectant mother. A speaker in any form (e.g. standalone loudspeaker, and mobile phone speaker) can also be used to provide an audio output.

The required power supplies and other peripheral components are not depicted in FIG. 2.

Although two receivers are shown, each will in practice comprise an array of transducer elements. The hand held probe may have a small number of elements for example of the order of 16 to 64. The transducer arrays may be implemented as CMUTs (capacitive micromachined ultrasound transducers). Another option is a thin-film ultrasound transducer array, for example with the transducers driven with low-voltage CMOS signals. The transmission pulse circuit 22 may comprise a low voltage pulse circuit. The fat body tissue is within the superficial area so that a large penetration depth is not required for the fat body tissue measurement. Thus, a relatively low transmission power is needed.

In use, the transmitter of the transceiver 12 sends a pulsed a low voltage to drive the two ultrasound transducers, one of which is in transmit then receive mode and the other of which only needs to be driven in receive mode.

The transceiver generates a pulsed ultrasound beam that transmits through the body tissue. Both transducers 12, 18 are used for receiving the echo wave from the tissue. The received ultrasound echoes are digitized and sent to the controller 20 for fat identification and thickness measurement. Both wired (e.g. USB) and wireless communicating methods (e.g. Bluetooth, Zigbee, WiFi) can be used for the signal communication between the hand held device, the controller 20 and the output device 34.

In use of the system 10, the user applies the system to the skin. The user is for example trained to be able to apply the system such that the desired 90 degree angle of incidence is achieved.

As will be explained below, the processing of the received sensor signals is based on the assumption that the signal transmitted and received by the transceiver 12 propagates normally in the body tissue. The propagation path is perpendicular to the fat body tissue layer 16a and the lean (muscle) body tissue layer 16b.

The operator is guided using the output device 34 to move the system 10 to assist in obtaining a correct position, as well as providing contact with the skin.

For the purposes of skin contact control, the hand held device may include a pressure sensor. This can be used to ensure that a pressure in a suitable range is applied. The suitable range ensures there is contact but not with such force that there is compression of the fat body tissue layer to alter the readings obtained. The tissue depth measurements are desired for body tissue at rest.

The system 10 is then held still, while a set of pulses (for example: three to 20) is sent to the same position for the subject in order to obtain a number of raw echo signals or DICOM ultrasound data. The use of multiple data samples, such as 5 to 20, enables averaging. Echo peaks are identified by signal processing and the timing of those peaks is determined.

The digitized ultrasound signal may be transferred to a mobile phone for the signal processing algorithm to estimate the ultrasound parameters and finally calculate the fat thickness. Thus, a stand-alone hand held device may be connected to a mobile phone to complete the overall system.

The system for example uses ultrasound, but it does not require the complexity of ultrasound imaging. The system enables the fat tissue depth to be determined and thereby the fat mass. This is based on a measurement of the acoustic speed of the fat body tissue and the acoustic speed of the lean body tissue, and measurement of the thickness of each layer. The ratio of fat tissue and lean tissue may then be obtained at multiple locations with high accuracy.

The system optionally also enables a check to be made if there is water retention, i.e. edema, in the pathway of the ultrasonic waves, by analysing acoustic properties of the soft tissue, including the fat tissue and lean tissue. These acoustic properties may include an acoustic attenuation coefficient or another representative parameter.

FIG. 3 shows the optical paths within the system.

The purpose of the reference patch 32 is to check if there is water retention. The reference patch 32 is for example: a patch made from silicone mixed with iron powder and has known acoustic properties. The thickness is for example between 1 cm and 2 cm to avoid near-field effects of the transducer. The reference patch 32 has similar acoustic properties to the fat tissue, lean tissue, or the whole soft tissue. These properties may change with the environmental factors, such as changes of temperature and humidity.

The acoustic property which is matched by the reference patch 32 can be an acoustic attenuation coefficient or its indicator:

$${R}_{ref} = 1 - \frac{{l}_{ref}}{{l}_{i}}$$

Ii is the input ultrasonic beam intensity and Iref is the echo intensity from the reference patch.

The matched acoustic properties can additionally or alternatively be an acoustic speed C_{ref}.

Taking the acoustic attenuation as an example, if there is water retention around the soft tissue (including both fat tissue and muscle tissue), the acoustic attenuation coefficient value is far smaller, since the acoustic attenuation coefficient of water at 1 MHz is 240 times smaller than that of the normal fat tissue.

Thus, the acoustic attenuation of the soft tissue layer, including fat mass and lean mass, is far smaller than for subjects without edema. The acoustic attenuation of the reference patch alone is selected to be comparable to soft tissue without edema. Thus, the acoustic attenuation in the body tissue alone is much smaller than in the reference patch.

In particular, it is identified if the measured acoustic attenuation coefficient for the body tissue is in the range of that of the reference patch. If the acoustic attenuation coefficient in within a predetermined range of that of the reference patch, the acoustic speed of the fat tissue and the lean tissue is determined from the echoes received at both receiver sites, and the fat/lean thickness or mass ratio may then be determined.

If the estimated acoustic attenuation coefficient for the body tissue is far smaller than that of the reference patch, it indicates that there is water retention, i.e. edema, around the soft tissue. The acoustic speed of the fat and lean tissue can still however be determined based on the echoes both receivers.

The reference patch also means that the temperature and humidity effect on the attenuation coefficient or acoustic speed can be removed by comparing the real-time measured value of R_{ref} for the reference patch.

As mentioned above, the reference patch is selected having similar acoustic properties to the soft tissue. However, the acoustic properties could change not only as a result of water from retention from edema, but also water from humidity and temperature. With no reference patch, the measured acoustic reflection (Rsoft) is a single obtained value and the presence of edema may be made by comparison of the value with a fixed reference.

However, by using a reference patch, the measured acoustic property will be a changing measurement, and when comparing the measured acoustic reflection (Rsoft) with the reference derived from the reference patch alone (Rref) more precise determination can be made as to whether edema has arisen.

As shown in FIG. 3, the transceiver 12 transmits ultrasonic waves with intensity Iᵢ, and receives echoes Iᵣ(t), in a direction perpendicular to the soft tissue. The receiver 18 is displaced by a distance y which is for example in the range 3 cm-5 cm and receives the reflected echoes. In this way, a first set of normal echoes, Iᵣ(τ₁), Iᵣ(τ₂) and Iᵣ(τ₃), will be received by the transceiver 12 while a second set of angled echoes, I′ᵣ(τ₂) and I′ᵣ(τ₃), will be received by the second receiver 18. The two sets of the echoes are the reflections of the propagating wave Iᵢ transmitted.

The thickness of the skin may be assumed to be far less than the thickness of fat body tissue and lean body tissue layers and also relatively constant. Based on this assumption, the acoustic speed and thickness of each layer can be calculated by using equations below:

$${C}_{fat} = \frac{y}{\sqrt{{( {τ}_{2}^{′} - {τ}_{2} + \frac{1}{2} {τ}_{1} )}^{2} - {( {τ}_{2} - {τ}_{1} )}^{2}}}$$

$${d}_{fat} = \frac{1}{2} {C}_{fat} \times ( {τ}_{2} - {τ}_{1} )$$

$${C}_{lean} = \frac{- Y + \sqrt{{Y}^{2} - 4 XZ}}{2 X}$$

$${d}_{lean} = \frac{1}{2} {C}_{lean} \times ( {τ}_{3} - {τ}_{2} )$$

$$X = {( {τ}_{3} - {τ}_{2} )}^{2} - {( 2 {τ}_{3}^{′} - {τ}_{3} )}^{2}$$

$$Y = ( 4 {τ}_{3}^{′} - 2 {τ}_{3} ) \sqrt{{{C}_{fat}^{2} ( {τ}_{2} - {τ}_{1} )}^{2} + 4 {y}^{2}}$$

$$Z = 4 {y}^{2} [ {( {τ}_{3} - {τ}_{2} )}^{2} - 1 ] - {{C}_{fat}^{2} ( {τ}_{2} - {τ}_{1} )}^{2}$$

Wherein, C_{fat} and Cₗₑₐₙ are the acoustic speed of the fat tissue and lean tissue layers, respectively. d_{fat} and dₗₑₐₙ are thickness of each layer. τ₁, τ₂, and τ₃ are times of echoes received by the first receiver of the transceiver 12, and τ′₂ and τ′₃ are times of echoes received by the second receiver 18.

These time values are shown in FIG. 3. There is a first reflection from the interface between the transceiver 12 and the reference patch 32, and this may be taken to be the reference time t=0. This reflection is shown as Iᵣ₀.

A next reflection is from the interface between the reference patch 32 and the skin surface, and this reflection interface is shown as Iᵣ(τ₁). A next reflection is from the interface between the fat tissue 16a and the lean tissue 16b, and this is shown as Iᵣ(τ₂). A next reflection is from the interface between the lean tissue 16b and the bone 16c, and this is shown as Iᵣ(τ₃).

The reflected signals received at the receiver 18 are shown as I′ᵣ(t). One reflection is from the interface between the fat tissue 16a and the lean tissue 16b, and this is shown as I′ᵣ(τ₂). A next reflection is from the interface between the lean tissue 16b and the bone 16c, and this is shown as I′ᵣ(τ₃).

The equations above are based on the transmission and reflection path to the transceiver 12 being normal to the skin surface, so that the path I′ᵣ(τ′₂) forms a right-angled triangle with the thickness d_{fat} and the separation y. Furthermore, the path I′ᵣ(τ′₃) is made up of two parts at different speeds and hence propagation angles. By extending the second part (shown as a dotted line in FIG. 3), a length ratio is obtained which is dependent on the speed ratio C_{fat}/Cₗₑₐₙ.

By combining all the paths shown using the basic approach that the length of each path is the time duration within that path and the speed (which is either Cₗₑₐₙ or C_{fat}), the equations above are derived. They enable the four parameters Cₗₑₐₙ, C_{fat}, dₗₑₐₙ and d_{fat} to be derived from the five timing measurements (τ₁, τ₂, τ₃, τ′₂, τ′₃) and the separation distance y.

To show this derivation in more detail, points A, B, C, C′, D and E are shown in FIG. 3. CD (for example) is used to denote the length of the path between points C and D.

τ₃−τ₂=2dₗₑₐₙ/Cₗₑₐₙ

τ₃−τ₁=2d_{fat}/C_{fat}+2dₗₑₐₙ/Cₗₑₐₙ

τ₃′−τ₁=d_{fat}/C_{fat}+dₗₑₐₙ/Cₗₑₐₙ+CD/Cₗₑₐₙ+DE/C_{fat}

To obtain distances CD and DE, the following relationships hold:

CD²=dₗₑₐₙ²+BD²

BD²+BC′²=C′D²

BC′=dₗₑₐₙ×C_{fat}/Cₗₑₐₙ (based on the angle change at D being dependent on the speed change)

(C′D+DE)²=(d_{fat}+BC′)²+y²

C′D/DE=BC′/d_{fat}

These relationships combine to form the equation above for C_{fat}, and for Cₗₑₐₙ the quadratic equation:

X·Cₗₑₐₙ²+Y·Cₗₑₐₙ+Z=0

With X, Y and Z as defined above.

In order to obtain a total body fat measure, measurements may be taken at multiple body locations. The fat tissue depth for each site may then be input to a regression equation to derive a total body fat measure. Such regression equations are known, and do not form part of this invention. A regional measurement may be used to estimate the overall body fat mass.

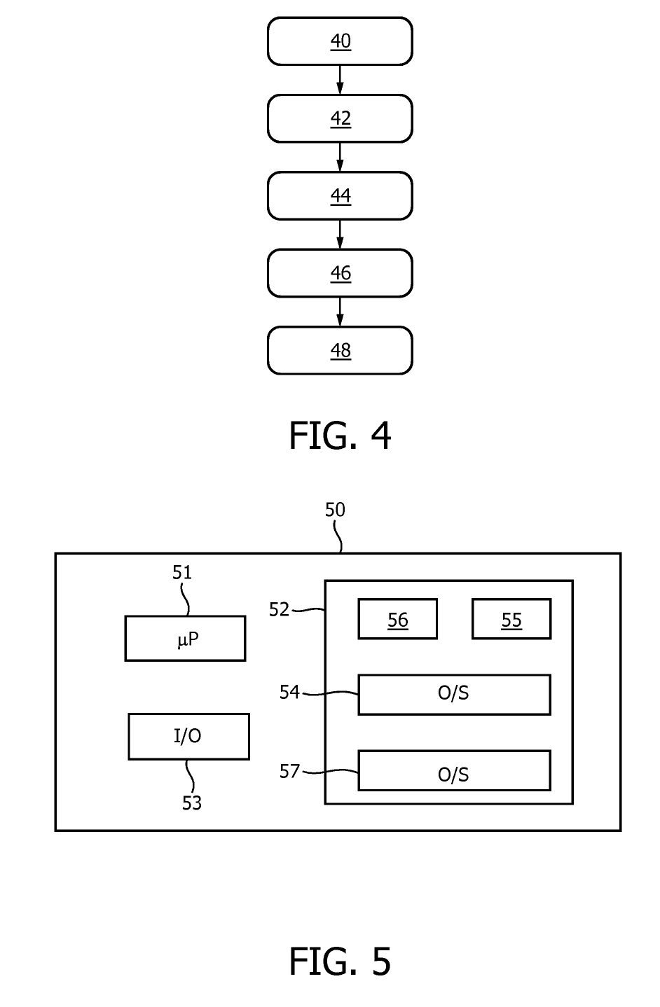

FIG. 4 shows a method for determining the fat and lean body tissue components. In step 40, a propagating wave is transmitted into the body tissue.

In step 42, a first set of normally directed reflections of the propagating wave is received from the body tissue at a first measurement site.

In step 44, a second set of angled reflections of the propagating wave is received from the body tissue at a second measurement site which is displaced from first measurement site.

In step 46, the times are determined at which reflections of the first and second sets are received.

In step 48 the determined times as well as the distance between the first and second measurement sites are processed, thereby to derive a fat body tissue depth dimension and a lean body tissue depth dimension.

The processing may use other input data, such as the gender, height and total weight of the subject, and these values may form part of the regression equation for determining a total fat body tissue mass and/or a ratio of fat body tissue mass to lean body tissue mass.

The system described above makes use of a controller for processing the collected data.

FIG. 5 illustrates an example of a computer 50 for implementing the controller or processor described above.

The computer 50 includes, but is not limited to, PCs, workstations, laptops, PDAs, cellphone, palm devices, servers, storages, and the like. Generally speaking, in terms of hardware architecture, the computer 50 may include one or more processors 51, memory 52, and one or more I/O devices 53 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 51 is a hardware device for executing software that can be stored in the memory 52. The processor 51 can be virtually any custom made or commercially available processor, a central processing unit (CPU), a digital signal processor (DSP), or an auxiliary processor among several processors associated with the computer 50, and the processor 51 may be a semiconductor based microprocessor (in the form of a microchip) or a microprocessor.

The memory 52 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 52 may incorporate electronic, magnetic, optical, and/or other types of storage media. Note that the memory 52 can have a distributed architecture, where various components are situated remote from one another, but can be accessed by the processor 51.

The software in the memory 52 may include one or more separate programs, each of which comprises an ordered listing of executable instructions for implementing logical functions. The software in the memory 52 includes a suitable operating system (O/S) 54, compiler 55, source code 56, and one or more applications 57 in accordance with exemplary embodiments.

The application 57 comprises numerous functional components such as computational units, logic, functional units, processes, operations, virtual entities, and/or modules.

The operating system 54 controls the execution of computer programs, and provides scheduling, input-output control, file and data management, memory management, and communication control and related services.

Application 57 may be a source program, executable program (object code), script, or any other entity comprising a set of instructions to be performed. When a source program, then the program is usually translated via a compiler (such as the compiler 55), assembler, interpreter, or the like, which may or may not be included within the memory 52, so as to operate properly in connection with the operating system 54. Furthermore, the application 57 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, the iOS development tool, and the like.

The I/O devices 53 may include input devices such as, for example but not limited to, a mouse, keyboard, scanner, microphone, camera, etc. Furthermore, the I/O devices 53 may also include output devices, for example but not limited to a printer, display, etc. Finally, the I/O devices 53 may further include devices that communicate with both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices may for example include the ultrasound transducers. The I/O devices 53 also include components for communicating over various networks, such as the Internet or intranet.

When the computer 50 is in operation, the processor 51 is configured to execute software stored within the memory 52, to communicate data to and from the memory 52, and to generally control operations of the computer 50 pursuant to the software. The application 57 and the operating system 54 are read, in whole or in part, by the processor 51, perhaps buffered within the processor 51, and then executed.

When the application 57 is implemented in software it should be noted that the application 57 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

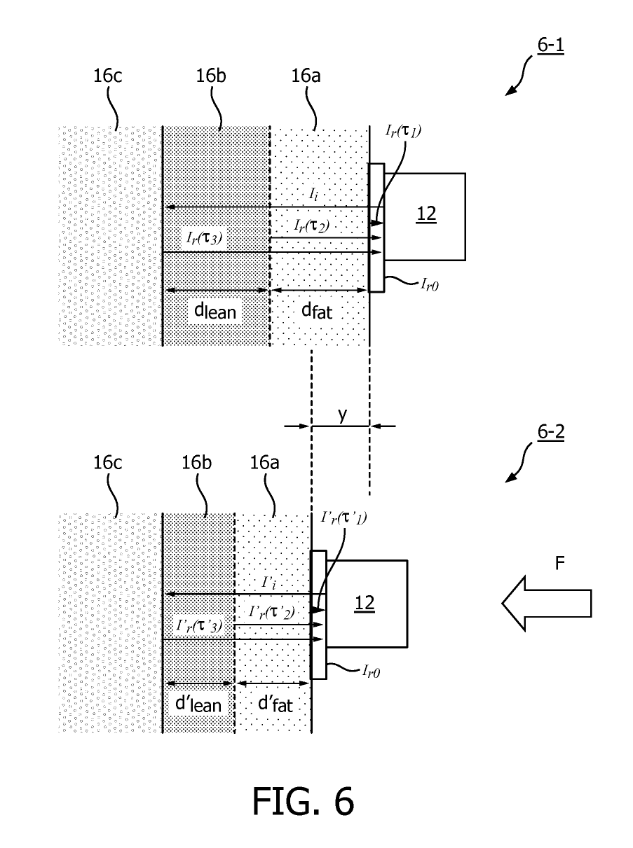

FIG. 6 illustrates a simplified system with identified acoustic paths in order to show how the timing calculations are performed. The simplified system may be implemented as a similar transceiver 12 illustrated in FIG. 2. It may further be provided with a reference membrane 32. FIG. 6 illustrates two phases 6-1 and 6-2 for the measurement procedure. Specifically, phase 6-1 shows the transceiver 12 at a first measurement site without deformation. At the first measurement site, a first propagating wave Iᵢ is transmitted by the transceiver 12 and a corresponding first set of normal echoes, Iᵣ(τ₁), Iᵣ(τ₂) and Iᵣ(τ₃), will be received by the transceiver 12. Then an external force F is applied to the transceiver 12 to push it in the direction perpendicular to the tissue layer. When the transceiver 12 reaches a second measurement site, which is at a distance y away from the first measurement site, a second propagating wave I′ᵢ is transmitted and correspondingly a second set of echoes, I′ᵣ(τ₁), I′ᵣ(τ₂) and I′ᵣ(τ₃), will be received by the second receiver 18. The two sets of the echoes are the reflections of the propagating waves Iᵢ and I′ᵢ respectively. The calculations of the depth and speeds follow the principle as for the embodiment illustrated in FIG. 3. Detailed derivations are provided as followings:

The acoustic speed and thickness of each layer can be calculated by using equations below:

$${d}_{fat} = 1 / 2 {C}_{fat} \times ( {τ}_{2} - {τ}_{1} )$$

$${d}_{fat}^{′} = 1 / 2 {C}_{fat} \times ( {τ}_{2}^{′} - {τ}_{1}^{′} )$$

$${d}_{lean} = 1 / 2 {C}_{lean} \times ( {τ}_{3} - {τ}_{2} )$$

$${d}_{lean}^{′} = \frac{1}{2 {C}_{lean}} \times ( {τ}_{3}^{′} - {τ}_{2}^{′} )$$

Wherein, C_{fat} and Cₗₑₐₙ are the acoustic speed of the fat tissue and lean tissue layers, respectively. d_{fat} and dₗₑₐₙ are thickness of each layer without tissue deformation. d′_{fat} and d′ₗₑₐₙ are thickness of each layer after deformation. τ₁, τ₂, and τ₃ are time of echoes received by Transducer in phase 6-1, whereas, τ₁′, τ₂′ and τ₃′ are time of echoes in phase 6-2 with deformation y.

Meanwhile, considering both fat and lean mass tissues are linear elasticity and according to the elastic principle, it can be derived that

$${d}_{fat} - {d}_{fat}^{′} + {d}_{lean} - {{d}_{lean}^{′}}_{} = y \\ {E}_{fat} \times \frac{{d}_{fat} - {d}_{fat}^{′}}{{d}_{fat}} = {E}_{lean} \times \frac{{d}_{lean} - {{d}_{lean}^{′}}_{}}{{d}_{lean}}$$

Wherein E_{fat} and Eₗₑₐₙ are modulus of fat and lean mass tissue and regarded as constant in this case. With the assumption that thickness of skin is far less than thickness of fat and lean mass layers and relative constant and two equations described above all, the acoustic speed of each layer can be calculated by:

$$\begin{matrix}{C}_{lean} = \frac{y [ 2 {E}_{lean} ( {τ}_{2} - {τ}_{1} ) - {E}_{fat} ( {τ}_{3} - {τ}_{2} ) ( {τ}_{2} - {τ}_{1} - {τ}_{2}^{′} + {τ}_{1}^{′} ) ]}{\begin{matrix}{E}_{lean} ( {τ}_{2} - {τ}_{1} ) ( {τ}_{3} - {τ}_{2} - {τ}_{3}^{′} + {τ}_{2}^{′} ) - \\ {E}_{fat} ( {τ}_{3} - {τ}_{2} ) ( {τ}_{2} - {τ}_{1} - {τ}_{2}^{′} + {τ}_{1}^{′} )\end{matrix}} & \\ {C}_{fat} = \frac{2 y - {C}_{lean} ( {τ}_{3} - {τ}_{2} - {τ}_{3}^{′} + {τ}_{2}^{′} )}{{τ}_{2} - {τ}_{1} - {τ}_{2}^{′} + {τ}_{1}^{′}} & \end{matrix}$$

Then thickness of each layer d_{fat} and dₗₑₐₙ can be determined as well.

This invention is of particular interest for home use or for use by non-experienced users. It means the fat layer can be identified and the fat thickness can be measured automatically. This reduces the user variability due to different levels of experience and at the same time increases the confidence of the fat measurement. The invention is of interest for obesity control by measuring fat thickness in daily use in the home, fitness assessment as part of a training regime, or for other health-related diagnosis.

Thus, it will be seen that the invention is applicable not only to monitoring during pregnancy but more generally for weight control and body composition assessment.

The example above is based on ultrasound. However, reflections of other waves may be employed, such as infrared (IR) or other electromagnetic waves which are able to penetrate to the depth of the fat layer to enable signal processing for fat detection.

The invention can be implemented on any ultrasound scanner (whether mobile, portable or fixed) as long as the system can provide a flow of ultrasound images or ultrasound raw RF signals.

This invention is thus an automated system and method for detection of fat tissue using non-ultrasound imaging devices, in particular for personal healthcare applications.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A system for measuring fat and lean body tissue components of body tissue, comprising:
a transceiver for transmitting a or more propagating wave normally into the body tissue, and receiving a first set of normally directed reflections of the propagating wave from the body tissue at a first measurement site;
a receiver for receiving a second set of reflections of the propagating wave from the body tissue at a second measurement site, which is displaced away from the first measurement site;
a timing arrangement for determining or estimating the times at which reflections of the first and second sets are received; and
a processor, which is adapted to:
control the transceiver;
receive a distance between the first measurement site and the second measurement site;
process the times determined or estimated by the timing arrangement and the distance received, thereby to derive a fat body tissue depth.

2. A system as claimed in claim 1, wherein the transceiver is adapted to transmit a first propagating wave at the first measurement site and the receiver is displaced from the transceiver along the body tissue for receiving the second set of angled reflections of the first propagating wave.

3. A system as claimed in claim 2, wherein the distance between the first measurement and the second measurement site is predetermined.

4. A system as claimed in claim 1, wherein the transceiver is adapted to transmit a first propagating wave at the first measurement site and transmit a second propagating wave at the second measurement site, and the receiver is the transceiver displaced at the second measurement site for receiving the second set of normal reflections of the second propagating wave.

5. A system as claimed in claim 4, wherein the distance between the first measurement site and the second measurement site is determined by a deformation sensor during the measurement.

6. A system as claimed in claim 1, wherein the processor is further adapted to derive one or more of:
a lean body tissue depth;
a depth ratio of lean and fat tissue depth;
a total fat mass.

7. A system as claimed in claim 1, wherein the receiver comprises an ultrasound receiver array.

8. A system as claimed in claim 1, further comprising a reference membrane over the transceiver such that it is in the wave path between the transceiver and the body tissue.

9. A system as claimed in claim 7, wherein the processor is adapted to determine whether or not the body tissue has a high water content indicative of water retention.

10. A system as claimed in claim 1, wherein the distance between the transceiver and the receiver is in the range 3 cm to 5 cm.

11. A system as claimed in claim 1, for monitoring a fat to lean body tissue ratio of an expectant mother during pregnancy.

12. A method for measuring fat and lean body tissue components of body tissue, comprising:
transmitting a or more propagating wave normally into the body tissue, and receiving a first set of normally directed reflections of the propagating wave from the body tissue through a first propagation path at a first measurement site;
receiving a second set of reflections of the propagating wave from the body tissue through a second propagation path at a second measurement site which is displaced from first measurement site;
determining or estimating the times at which reflections of the first and second sets are received; and
receiving a distance between the first measurement site and the second measurement site;
processing the times determined or estimated by the timing arrangement and the distance received, thereby to derive a fat body tissue depth.

13. A method as claimed in claim 12, comprising transmitting a first propagating wave at a first measurement site and receiving a second set of angled reflections of the first propagating wave at the second measurement site.

14. A method as claimed in claim 12, comprising transmitting a first propagating wave at a first measurement site and a second propagating wave at a second measurement site, and receiving the second set of normal reflections of the second propagating wave.

15. A computer program comprising code means which is adapted, when said program is run on a computer, to perform the method of claim 12.
